(19) United States
(12) Patent Application Publication
(10) Pub. No.: US 2008/0081359 A1
(43) Pub. Date: Apr. 3, 2008

(54) METHODS FOR PRODUCING MUTANT MICROBES USEFUL FOR PRECIOUS METAL AND BIOENERGY PRODUCTION

(75) Inventors: JOE E. CHAMPION, Washington, UT (US); Raymond Owyang, San Francisco, CA (US)

Correspondence Address: RAYMOND OWYANG, 20 WOOD STREET, SAN FRANCISCO, CA, 94118, US

(73) Assignee: BIOMETAL L.L.C., SAN FRANCISCO, CA (US)

(21) Appl. No.: 11/862,424

(22) Filed: Sep. 27, 2007

Related U.S. Application Data
(60) Provisional application No. 60/827,429, filed on Sep. 29, 2006.

(51) Int. Cl. C12P 3/00; C10G 32/00; C12N 1/00; C12N 15/01

(52) U.S. Cl. 435/168; 435/255.2; 435/281; 435/441

(57) ABSTRACT

A mutant microbe that generates trace amounts of gold on silver, and uses of the mutant microbe for recovering precious metals and producing biofuels and oil products are described. According to an exemplary embodiment, the mutant microbe is produced by placing metallic silver in an aqueous solution, and adding a species of Saccharomyces to the aqueous solution such that when the species of Saccharomyces comes in contact with the metallic silver, at least a portion of the species of Saccharomyces transforms into the mutant microbe that interacts with the metallic silver and forms a layer comprising a trace amount of nano gold particles on the metallic silver.

### CROSS-REFERENCE TO RELATED APPLICATIONS

This application claims priority to a provisional application entitled “METHODS FOR PRODUCING MUTANT MICROBES USEFUL FOR PRECIOUS METAL RECOVERY AND BIO-ENERGY PRODUCTION,” Ser. No. 60/827,429, filed Sep. 29, 2006, which is herein incorporated in its entirety by reference.

### COPYRIGHT NOTICE

A portion of the disclosure of this patent document contains material which is subject to copyright protection. The copyright owner has no objection to the facsimile reproduction by anyone of the Patent and Trademark Office patent file or records, but otherwise reserves all copyright rights whatsoever.

### FIELD OF THE INVENTION

The present invention relates to methods of mutation of yeast of the genus Sacchromyces with metallic silver. The mutant microbes carry out biological transmutation in coating silver with a trace amount of nano gold particles. The mutated microbes are useful in a number of applications including the recovery of precious metal values from mineral ores and the production of biofuels and oil products using both inorganic and organic matter as nutrient sources.

### BACKGROUND

Biological Transmutation. Biological transmutation can be defined as a nuclear transmutation occurring in living organisms. Generally, the phenomenon is not accepted by mainstream science, which argues that transmutations are only possible in high-energy nuclear reactions. Such reactions are physically impossible in biological systems, as the amount of energy used in such a manner would be fatal within a several-kilometer radius. Proponents respond that evidence shows that transmutations do occur, and that the lack of a theoretical model adequately explaining the mechanisms involved (that is, without the emission of deadly amounts of energy) does not render that evidence invalid. The most prominent defender of the existence of biological transmutations is the French scientist Corentin Louis Kervran, who investigated discrepancies between the dietary or environmental intake of elements such as calcium, potassium or magnesium by various organisms and the quantities they hold or excrete. For instance he investigated the source of calcium chickens use for their eggshells, and concluded that they probably convert the calcium from dietary potassium.

Applicants have discovered mutant microbes obtained by treating microbes in aqueous solution with silver. The mutant microbes coat silver with a thin layer of a yellow material comprising a trace amount of nano gold particles by a biological transmutation process. Allotropic silver is yellow. But spectroscopic x-ray analysis and conventional metallurgical fire assay methods show the yellow material deposited on silver by the mutant microbes comprises trace amounts of nano gold particles.

Nanotechnology. Nanoparticles are of great scientific interest as they are effectively a bridge between bulk materials and atomic or molecular structures. A bulk material should have constant physical properties regardless of its size, but at the nano-scale this is often not the case. Size-dependent properties are observed such as quantum confinement in semiconductor particles, surface plasma resonance in some metal particles and superparamagnetism in magnetic materials.

The properties of materials change as their size approaches the nanoscale and as the percentage of atoms at the surface of a material becomes significant. For bulk materials larger than one micrometer the percentage of atoms at the surface is minuscule relative to the total number of atoms of the material. The interesting and sometimes unexpected properties of nanoparticles are not partly due to the aspects of the surface of the material dominating the properties in lieu of the bulk properties.

Nanoparticles exhibit a number of special properties relative to bulk material. For example, the bending of bulk copper (wire, ribbon, etc.) occurs with movement of copper atoms/clusters at about the 50 nm scale. Copper nanoparticles smaller than 50 nm are considered super hard materials that do not exhibit the same malleability and ductility as bulk copper. The change in properties is not always desirable. Ferroelectric materials smaller than 10 nm can switch their magnetization direction using room temperature thermal energy, thus making them useless for memory storage. Suspensions of nanoparticles are possible because the interaction of the particle surface with the solvent is strong enough to overcome differences in density, which usually result in a material either sinking or floating in a liquid. Nanoparticles often have unexpected visible properties because they are small enough to confine their electrons and produce quantum effects. For example gold nanoparticles appear deep red to black in solution.

Applicants have discovered that mutant microbes obtained by mutating microbes in aqueous solution with metallic silver deposit a thin layer of nano gold atoms and particles onto silver by a biological transmutation process.

Microbes for Precious Metal Recovery. The uses of microbes for recovering precious metals from mineral ores are known. Precious metals are frequently occluded, encapsulated, bonded and/or alloyed in mineral ores and are not amendable to conventional recovery methods. For example, gold often occurs as finely disseminated sub-microscopic particles within a refractory sulfide host of pyrite or arsenopyrite. Bio-oxidation is used to liberate the gold occluded within the sulfide host. A number of processes for bio-oxidizing the sulfide minerals are known in the art. One known method of bio-oxidizing the metal sulfides in an ore is to use bacteria, such as Thiobacillus ferrooxidans, sulfolobus, acidianus species and facultative-thermophilic bacteria in a microbial pretreatment.

Applicants have discovered that the mutant microbes are useful for recovering precious metals from mineral ores and the biomass of dead mutant microbes of the invention.

Microbes for Biofuel Production. The use of microorganisms to produce methane and ethanol from organic matter are known in the art. For example, ethanol for use as fuel and in alcoholic beverages is produced by fermentation of sugar by certain species of yeast (most importantly, Saccharomyces cerevisiae).

Applicants have discovered that the mutant microbes of this invention are useful for production of biofuels and oil products from sedimentary organic matter and biomass, including heavy oil.

### SUMMARY OF THE INVENTION

According to an exemplary embodiment, a mutant microbe that generates trace amounts of gold on silver, and uses of the mutant microbe for recovering precious metals and producing biofuels and oil products are described. According to an exemplary embodiment, the mutant microbe is produced by placing metallic silver in an aqueous solution, and adding a species of Saccharomyces to the aqueous solution such that when the species of Saccharomyces comes in contact with the metallic silver, at least a portion of the species of Saccharomyces transforms into the mutant microbe that interacts with the metallic silver and forms a layer comprising a trace amount of nano gold particles on the metallic silver.

According to another exemplary embodiment, a method of producing a mutant microbe used for generating trace amounts of gold particles on metallic silver includes placing metallic silver in an aqueous solution and adding a species of Saccharomyces to the aqueous solution such that when the species of Saccharomyces comes in contact with the metallic silver, at least a portion of the species of Saccharomyces transforms into a mutant microbe that interacts with the metallic silver and forms a layer comprising a trace amount of nano gold particles on the metallic silver.

According to another exemplary embodiment, a method for producing precious metals includes placing metallic silver in an aqueous solution, adding a species of Saccharomyces to the aqueous solution such that when the species of Saccharomyces comes in contact with the metallic silver, at least a portion of the species of Saccharomyces transforms into a mutant microbe that includes clusters of precious metal atoms within its cytoplasm and forms a layer comprising a trace amount of nano gold particles on the metallic silver, and recovering the cluster of precious metal atoms from the mutant microbe.

According to another exemplary embodiment, a method for producing precious metals includes placing metallic silver in an aqueous solution, adding a species of Saccharomyces to the aqueous solution such the when the species of Saccharomyces comes in contact with the metallic silver, at least a portion of the species of Saccharomyces transforms into a mutant microbe that interacts with the metallic silver and forms a layer comprising a trace amount of nano gold particles on the metallic silver, and contacting a mineral ore with an aqueous solution including the mutant microbe.

According to another exemplary embodiment, a method for producing oil products from a sedimentary organic rock, heavy oil and/or a biomass includes placing metallic silver in an aqueous solution, adding a species of Saccharomyces to the aqueous solution such the when the species of Saccharomyces comes in contact with the metallic silver, at least a portion of the species of Saccharomyces transforms into a mutant microbe that interacts with the metallic silver and forms a layer comprising a trace amount of nano gold particles on the metallic silver, and contacting at least one of the sedimentary organic rock, the heavy oil and the biomass with the mutant microbe.

### BRIEF DESCRIPTION OF THE DRAWINGS

Objects and advantages of the present invention will become apparent to those skilled in the art upon reading this description in conjunction with the accompanying drawings, in which like reference numerals have been used to designate like elements, and in which:

FIG. 1 is an image generated by a scanning electron microscope (SEM) depicting mutant microbes at 10,000× magnification;

FIG. 2 is an image generated by an SEM depicting mutant microbes at 20,000× magnification;

FIG. 3 is an image generated by a scanning electron microscope (SEM) depicting Silver Granules Coated with Yellow Material at 1000× magnification;

FIG. 4 is an image generated by a scanning electron microscope (SEM) depicting Mineral Ore before Biotreatment at 1000× magnification;

FIG. 5 is an image generated by a scanning electron microscope (SEM) depicting Mineral Ore after Biotreatment at 10,000× magnification; and

FIG. 6 is an image generated by a scanning electron microscope (SEM) depicting Biomass of Dead Mutant Microbes at 1000× magnification.

### DETAILED DESCRIPTION

Microbes are well known, commercially available and widely used in industrial microbiology. According to one embodiment, the microbes used herein are single-celled and non-pathogenic. Known industrial microbes include the genus of Saccharomyces and Schizosaccharomyces. Preferred species of Sacchromyces include the species: S. cerevisiae, S. bayanus, S. boulardii, S. pastorianus, S. uvarum, S. carlsbergensis, S. ellisoidesu, S. exiguus, S. fragilis, S. chevalieri, S. chodati, S. diastaticus and S. rouxii. Preferred species of Schizosaccharomyces is Schizosaccharomyces pombe. Other commonly known and industrial microbes that can be used include Aspergillus niger, Aspergillus orzae, Ashbya gossypii, Streptomyces species, Bacillus thuringiensis, Rhizobium, Bradyrhizobium, Bacillus subtilis, Corynebacterium glutamicum, Leuconostoc mesenteroides, Streptodornase pyogenes, and Thiobacillus ferrooxidans. The genus Saccharomyces is the preferred fungi for use in this invention. The preferred species are S. cerevisiae and S. carlsbergensis. These yeasts are commercially available.

In the embodiments described, natural microbes, such as those present in alkali flat lake bed deposits in Franklin Lake or Alkali Flat in Inyo County, Calif., can be used. Other natural bacteria are dormant bacteria in dried lakebeds and seabeds such as the Great Salt Lake deposits of Utah and the Winnemucca Lake deposits of Nevada.

In an exemplary embodiment, the microbes are mutated from industrial microbes and natural ancient microbes in mineral ores by a process that includes contacting the microbe(s) in an aqueous solution with metallic silver. The metallic silver can be particles, grains, granules, and/or bars, ranging in size from 1 micron particles to silver bars of 10 kilograms or more. Colloidal silver solutions with colloidal silver in aqueous solution ranging in concentration from 1 ppm to 10 ppm and particle sizes from 1 nanometer to 1 micron can be used. In one embodiment, metallic silver of 1 micron or more in size to silver bars of 10 kilogram or more is used. The contacting can be done without agitation, but preferably with mechanical agitation, air agitation (pumping air or oxygen into the aqueous solution) and/or pumping and passing the microbe in an aqueous nutrient solution through columns, tubes and tanks containing metallic silver. The mutation may be performed at temperatures ranging from 20 degrees centigrade to 90 degrees centigrade, preferably at temperatures ranging from 30 degrees centigrade to 50 degrees centigrade.

The aqueous solution may contain sufficient nutrients to support microbial growth. The useful nutrients are both inorganic and organic compounds commonly used to grow and nourish microbes. Inorganic nutrients include nitric acid, ammonium nitrate, ammonium chloride, ammonium sulfate, sodium nitrate, sulfur, sodium sulfide, sodium chloride, sodium bicarbonate, sodium phosphate, potassium phosphate, ferric chloride, calcium chloride, and ammonium phosphate. Organic nutrients include microbial biomass, glucose, dextrose, sodium acetate, amino acids, and purines. Microbial biomass may be dead microbes being used for mutation. Vitamins that can be included in the nutrient solution include pyridoxine, pyridoxamine-HCl, riboflavin, thiamine, niacin, pantothenic acid, p-aminobenzoic acid, folic acid, and biotin. Very small amounts of trace elements such as iron, copper, molybdenum and zinc can also be provided in the nutrient solution. Useful nutrients can also be mineral ores used for recovery of metals and sedimentary organic matter and rocks used for liberation of oil products.

In one embodiment, the mutation process is performed in an aqueous solution and the biomass from dead microbes is used as nutrients until the mutant microbes reach a density of 1% or more. Pressure is not critical and can be atmospheric, below atmospheric and/or above atmospheric.

The mutation can be conducted in aerobic or anaerobic conditions. However, the mutation is preferably conducted in the presence of nitrogen, carbon dioxide, and oxygen in the atmosphere. Oxygen can be provided chemically, for example, with hydrogen peroxide, or as a gas from pressurized vessels.

The mutant microbes may be single-celled or multi-celled microbes. They are usually round, but can be oval, elongated or flattened on one side. They have also been observed to be rod-shaped bacillus. They range in size from 0.20 to 2.0 micron in diameter and 2 to 8 micron in length. They have been observed to divide by budding and binary cell division.

Once created, the mutant microbes are stored and maintained by conventional microbiology techniques. A healthy mutant microbe can be isolated and grown to microbe colonies of 1% to 5% by weight in nutrient solutions. Nutrients can be inorganic, including nitric acid, ammonium nitrate, ammonium chloride, ammonium sulfate, sulfur, sodium sulfide, sodium nitrate, sodium chloride, sodium bicarbonate, sodium phosphate, potassium phosphate, ferric chloride, calcium chloride, and ammonium phosphate, and organic, including microbial biomass, glucose, dextrose, sodium acetate, amino acids, and purines.

Vitamins that can be included in the nutrient solution include pyridoxine, pyridoxamine-HCl, riboflavin, thiamine, niacin, pantothenic acid, p-aminobenzoic acid, folic acid, and biotin. Microbial biomass may be dead microbes being used for mutation. Very small amounts of trace elements such as iron, copper, molybdenum and zinc can also be provided in the nutrient solution. When it is desirable to grow the mutant microbes on a solid medium, a solidifying agent such as agar (a complex polysaccharide derived from a marine alga) is added to the media.

Silver and Ultraviolet Germicidal Irradiation. Alternatively or in addition, microbes can be mutated by ultraviolet germicidal irradiation and by a combination of metallic silver and UV irradiation. Ultraviolet (UV) light is electromagnetic radiation with wavelengths shorter than visible light. Ultraviolet can be separated into various ranges, with near range (less than 280 nm/2800 Angstrom) considered “germicidal UV”. In one embodiment, UV in the range of 280 nm to 390 nm is used to expose microbes. Forced flow of air or water can be used to agitate the microbial solution to ensure exposure to the UV radiation. The mutation using UV light may be done at temperatures ranging from 20 degrees centigrade to 80 degrees centigrade, and preferably at temperatures ranging from 30 degrees centigrade to 50 degrees centigrade. In one embodiment, the UV irradiation and the silver germicidal mutation process are done together.

Silver and Electromagnetic Field. In another embodiment, microbes can be mutated by conducting the mutation process in an electromagnetic field. For example, the mutation process can be conducted in a bioreactor with means to provide an electromagnetic field. The electromagnetic field can be provided by wrapping the bioreactor, such as a beaker, with copper wire and running an AC current of about 5 to 10 amps through the copper wire. In large bioreactors, for example, 1,000 liters to 10,000 liters, the microbial solution can be pumped through a glass column wrapped with copper wire for current flow. The current can be provided with a variable transformer.

Carbon and Iron Arcs. In another embodiment, the mutation process is conducted in a bioreactor equipped with carbon or iron arcs. The arcs can be provided with means for generating a voltage of about 5 to 10 volts between the carbon or iron arcs.

The mutant microbes that are mutated by the methods described have been observed to contain clusters of precious metal atoms within the cytoplasm of the cell. The metals within the cytoplasm are observed as clusters, curved bands and circular rings of metal atoms. Some mutated microbes have sets of two to ten concentric rings. Using an electron scanning microscope, the clusters, bands, and concentric rings of metal atoms within the cellular structure have been identified as silver and gold atoms and particles.

The mutant microbes can be identified and characterized by their ability to coat silver granules with a coating of a trace amount of nano gold particles. The coating process is done by contacting an aqueous solution of the mutant microbes with metallic silver. The amount of coating onto the silver ranges from 500 ppm to 1000 ppm and is dependent on the contact time, contact temperature and density of the microbial solution. The temperature ranges from 20 C to 90 C. The contact time ranges from one hour to 100 hours. With a high microbial density of 3 to 5% by weight, the contact time is about 1 to 4 hours. The amount of nano gold particles in the coating is approximately 100 ppm to 200 ppm based on X-ray diffraction analysis and scanning electron microscope analysis.

Mineral Ores. For purposes of this disclosure, the term “mineral” or “mineral ore” means a composition that comprises precious metal values. Thus, a mineral may be a mined mineral, ancient seabed deposit, ancient lakebed deposit, black sands, an ore concentrate, metal bearing sea water, and waste products, such as mining tails, industrial waste water, oil well brine, coal tars, oil shales, tar sands, and oil sands. Useful minerals contain trace amounts of precious metals. Trace amount means the detection limit or below detection limits of conventional assay procedures such as fire assay, AAS (atomic adsorption spectroscopy), ICP-MS (inductive coupled plasma-mass spectrometer), ICP-AES (atomic emission spectroscopy) and other spectroscopic instrumentation commonly used in analytical laboratories. Some spectroscopic methods can detect as little as 1 ppt (part per trillion) to 0.1 ppb (part per billion). Preferred mineral ores have from about 1 ppb to 100 ppm of precious metals.

Digestion and Metal Recovery. In one embodiment, the digestion and biotreatment of the mutant microbes with the mineral ores are conducted in commercially available bioreactors consisting of a reactor having an agitation means. The agitation means can be mechanical stirring with a flat bladed impeller, percolation column, or air agitated pachuca reactor. The bioreactor can have air intake means, sterilization means, harvesting means, heating and/or cooling means, temperature controller means, pH controller means, filtration means and pressure controller means. All these features of bioreactors are known and commercially available in the biotechnology industry.

The digestion of the mineral ores by the mutant microbes can also be done by heap leaching techniques. In heap bio leaching techniques, a large body of mineral ore is treated with mutant microbes in nutrient solution in large contaminant ponds with no agitation and/or only occasional agitation. Generally, the contact time for heap type bio treatment is substantially longer than the agitated bioreactors, and range from 10 days to 100 days.

The mineral ore can be milled and ground to 10 mesh to 300 mesh, preferably 100 to 200 mesh. The minerals useful in the invention are low grade and high grade precious metal minerals. Low grade minerals contain from 1 ppb to 1 ppm of a precious metal, preferably gold and silver. High grade minerals contain from 2 ppm to 100 ppm. Bio treatment temperature ranges from 15 degrees centigrade to 50 degrees centigrade, preferably from 20 degrees to 30 degrees centigrade. pH can be acidic (pH 1 to 3) or basic (pH 9 to 12), although slightly acidic (pH 4) to slightly basic (pH 8) pH ranges are preferred. The most preferred pH ranges are the neutral range of from pH 6.5 to pH 7.5.

Microbe concentration is not critical. At low microbe concentration, the contact duration is generally longer to allow the microbe to grow and multiply. However, microbe concentration should not exceed the maximum microbe concentration that the nutrient solution can sustain. Contact time can vary from a few hours to several weeks and depends in part on the type and mesh size of the mineral ore digested. Contact time ranges can be from 1 day to 30 days, more preferably from 1 day to 10 days. The ratio of mineral ore to nutrient solution is also not critical. Generally for ease of agitation, the ratio of mineral ore to microbe/nutrient solution, hereinafter, also referred as the pulp density, varies from 10% by weight mineral ore in the nutrient solution to 50% by weight mineral ore in the nutrient solution.

The digestion can be conducted in aerobic or anaerobic conditions. However, the mutation is preferably conducted in the presence of oxygen, nitrogen and carbon dioxide in the atmosphere. Oxygen can also be provided chemically, for example, with hydrogen peroxide, or as a gas from pressurized vessels.

Nutrients can also be provided in the digestion of mineral ore to support growth of the mutant microbes. Nutrients can be inorganic, including nitric acid, sulfur, ammonium nitrate, ammonium chloride, ammonium sulfate, sodium nitrate, sodium chloride, sodium bicarbonate, sodium phosphate, potassium nitrate, potassium phosphate, ferric chloride, calcium chloride, and ammonium phosphate, and organic, including glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Very small amounts of traces elements such as iron, copper, molybdenum and zinc can also be provided in the nutrient solution.

Nutrients can be added to the digestion as needed to maintain the sufficient mutant microbes for microbial growth and metal liberation. Microbial growth can be measured by conventional direct methods such as plate count, serial dilution, pour plates, spread plates and direct microscope count. Microbial growth can also be measured by indirect methods such as turbidity and metabolic activity.

After digestion with the mutant microbes, the recovery of metal from the mineral ore and microbial solution can be performed by conventional metallurgical methods such as smelting, leaching, electrolysis, resins and other methods known to those skilled in art of metallurgy. In another embodiment, the precious metals in the mutant microbes or biomass of dead mutant microbes can be recovered by methods described for recovery of precious metals from mineral ore.

Sedimentary Organic Matter and Rocks. According to other exemplary embodiments, the mutant microbes are used for producing oil products and biofuels from a sedimentary organic matter and rock. Suitable sedimentary organic matter includes coal, bituminous coal, sub-bituminous coal, lignite, bitumen, coal tar, fly ash, shale, tar sands and oil sands. Sedimentary organic matter that contains a high content of sulfur and sulfides can be used. Oil shale is found in the Western United States, especially the states of Utah, Wyoming, and Colorado, and oil sands found in northern Alberta, Canada. Oil shale is a general term applied to a group of rocks rich enough in organic matter (called kerogen) to yield oil products upon distillation. Oil sands, also referred to as tar sands or bituminous sands, are a combination of clay, sand, water and bitumen. Sedimentary organic matter and rocks generally contain from 1% to 99% organic matter, preferably 10 to 90% organic matter.

Biomass. According to other exemplary embodiments, the mutant microbes are used for producing oil products and biofuels from biomass. Biomass is any recently living organisms or their metabolic by products. Biomass can be of plant or animal origin. Useful biomass include agricultural residues such as rice straw, stover, wheat straw; agricultural wastes such sugarcane bagasse, rice hulls, corn fiber, sugar beet pulp, citrus pulp, citrus peels; forestry wastes such as hardwood and softwood thinning and hardwood and softwood residues from timber operations; and wood wastes such as saw mill waste and pulp and paper mill waste; urban wastes such as paper fraction of municipal solid waste; urban wood waste and urban green waste, and dedicated crops such as switchgrass, hybrid poplar wood, grains, maiden grass. Simple sugars or monosaccharides, such as glucose, fructose, and dextrose can also be used. Preferred biomass feed stocks are plant cellulosic biomass—that is, biomass composed primarily of inedible plant fibers having cellulose and hemicellulose as a prominent component. The biofuel produced depends on the biomass feedstock, and include methanol, ethanol, propanol, butanol, mixed alcohols, biogases. In biorefining and bioconverting embodiments, the mutant microbes are used to convert, refine and degrade heavy oils, bitumen, asphalt and tar to lower molecular weight and density petroleum products.

Heavy Oil and Enhanced Oil Recovery. A preferred form of biomass is heavy oil. The mutant microbes can be used for bioconversion, biorefining and biodegradation of heavy oil that is too viscous to ship through a pipeline to lighter oil that can be shipped in pipelines. Examples are surface heavy oil deposits, heavy oil recovered from oil sands and oil shale and heavy oil in oil wells and in depleted and abandoned oil wells. The mutant microbes can be injected in the oil wells with water and/or steam commonly used for secondary and enhanced oil recovery. Once the heavy oil is biodegraded to oil of a lower viscosity, it can be pumped from the oil well and transported in pipelines. The microbes can also be used to bioconvert surface heavy oil deposits to lighter oil products that can also be shipped in pipelines.

Biorefining, bioconversion and biodegrading methods. The digestion and biotreatment of the mutant microbes with biomass, heavy oil and sedimentary organic matter is conducted in commercially available bioreactors consisting of a reactor having an agitation means. The agitation means can be mechanical stirring with a flat bladed impeller, percolation column, air agitated Pachuca reactors, and continuous flow stirred tank reactors. The bioreactor can have air intake means, sterilization means, harvesting means, heating and/or cooling means, temperature controller means, pH controller means, filtration means and pressure controller means. All these features of bioreactors are known and commercially available in the biotechnology, biorefining and biomining industry. The digestion the biomass and sedimentary organic matter by the mutant microbes can also be done by heap leaching techniques. In heap bio leaching techniques, a large body of mineral ore is placed in a heap or dump where is it irrigated and treated with mutant microbes. Generally, the contact time for heap type bio treatment is substantially longer than the agitated bioreactors, and range from 10 days to 100 days.

The biomass and sedimentary organic matter can be milled and ground to particles in the range of 10 mesh to 300 mesh, preferably 100 to 200 mesh.

Bio treatment temperature ranges from 15 degrees centigrade to 90 degrees centigrade, and preferably range from 20 degrees to 50 degrees centigrade. pH can be acidic (pH 1 to 3) or basic (pH 9 to 12), although slightly acidic (pH 4) to slightly basic (pH 8) pH ranges are preferred. The most preferred pH ranges are the neutral range of from pH 6.5 to pH 7.5.

Microbe concentration is not critical. At low microbe concentration, the contact duration is generally longer to allow the microbe to grow and multiply. However, microbe concentration should not exceed the maximum microbe concentration that the nutrients can sustain.

Contact time can vary from a few hours to several weeks and depends in part on the type and mesh size of the biomass and sedimentary organic matter digested. Contact time ranges can be from 1 day to 30 days, more preferably from 1 day to 10 days.

The ratio of biomass and sedimentary organic matter to microbial solution is also not critical. Generally for ease of agitation in stirred-tanks, the ratio of biomass or sedimentary organic matter to microbe solution varies from 10% by weight mineral ore in the microbial solution to 50% by weight mineral ore in the microbial solution, and preferably about 15% by weight to 25% by weight. The digestion can be conducted in aerobic or anaerobic conditions. However, the mutation is preferably conducted in the presence of oxygen, nitrogen and carbon dioxide in the atmosphere. Oxygen can also be provided chemically, for example, with hydrogen peroxide, or as a gas from pressurized vessels.

Nutrients can also be provided in the digestion of biomass, heavy oil or sedimentary organic matter to support growth of the mutant microbes. Nutrients can be inorganic and/or organic.

Suitable media for growing mutant microbes and producing precious metals are nutrient media containing 1 to 10% by weight nitric acid. Vitamins that can be included in the nutrient solution include pyridoxine, pyridoxamine-HCl, riboflavin, thiamine, niacin, pantothenic acid, p-aminobenzoic acid, folic acid, and biotin. Very small amounts of traces elements such as iron, copper, molybdenum and zinc can also be provided in the nutrient solution. The biomass and organic matter present in sedimentary organic matter also serve as nutrients.

Nutrients can be added to the digestion as needed to maintain the sufficient mutant microbes for microbial growth. Microbial growth can be measured by conventional direct methods such as plate count, serial dilution, pour plates, spread plates and direct microscope count. Microbial growth can also be measured by indirect methods such as turbidity and metabolic activity.

Metals and Biofuels Co-Production. With sedimentary organic matter and fossil fuels containing precious and base metals, both metals and gaseous and liquid petroleum and metals are liberated and produced by the mutant microbes. After bio treatment, petroleum products are recovered and refined by conventional petroleum processes and the precious and base metals are recovered by conventional precious metal beneficiation processes such as electrowinning and/or dcyanidation. In one embodiment the mutant microbes are used to treat a mixture of a metal mineral ore and sedimentary organic matter and/or a fossil fuel. In this embodiment, the liquid oil products released from the sedimentary organic matter captures and floats the metals released from the metal mineral ore by a process similar to flotation or froth flotation processes used in the mining industry. The bio treatment procedures used for bio-energy and bio-fuel production are the same as the bio treatment and digestion procedures used for liberation of metals and are known industrial bio tech processing procedures.

Electromagnetic Field, Carbon Arcs and Iron Arcs. In other embodiments, the biotreatment of minerals ores, sedimentiary organic matter and biomass can be conducted in bioreactors equipped with an electromagnetic field, an electromagnetic field and carbon arcs and an electromagnetic field and iron arcs.

Fire assaying and cupellation are described by C. W. Ammen, Recovery and Refining of Precious Metals, second edition 1993, Chapter 12, pp 302-329.

### EXAMPLES

The above embodiments and other objects, features and advantages of this invention will become apparent to those skilled in the art from the following examples and descriptions of the embodiments. The examples are presented to one of ordinary skill in the art to make and use the invention and are provided in the context of a patent application and its requirements. Various modifications to the preferred embodiments and the generic principles and features described herein will be readily apparent to those skilled in the art. Thus, the present invention is not intended to be limited to the embodiments shown, but is to be accorded the widest scope and consistent with the principles and features described herein.

### Mutation Examples

### Example 1

### Silver Mutation Method

A Petri dish containing 2 grams of silver granules, 4 grams of Saccharomyces cerevisiae and 10 ml of distilled water were stirred occasionally over a ten day period. A small sample of aqueous solution was then placed on a glass slide with cover. The slide was then examined under a Meiji binocular biological optical microscope. Live microbes could be observed with a dense band of metal atoms within its cellular structure at magnifications as low as 500×. Some of the live microbes were then examined with a LEO electron scanning microscope (SEM) at 10,000× and 20,000× magnifications. Clear bands of metal atoms in concentric rings could be observed. Using an EDAX x-ray spectrometer, the metal bands were determined to be metallic.

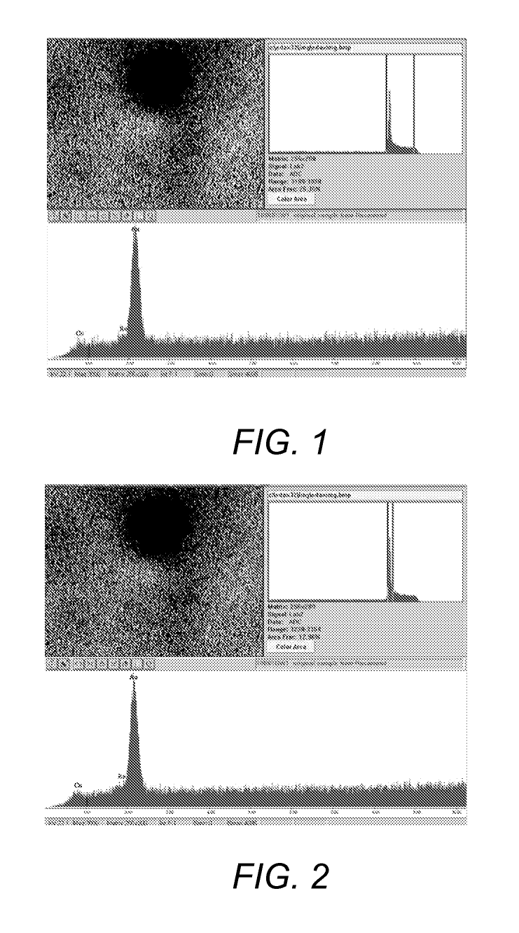

FIG. 1 and FIG. 2 are images of the mutant microbes viewed with the SEM. The SEM used was a Leo model 1430VP with tungsten filaments and an Edax 10 millimeter sapphire x-ray diffraction detector. The optical microscope used is Mieji lightfield model number 5500 with an Infinity 1100 camera.

### Example 2

### Silver Method

A 200 gallon tank reactor about 2 feet wide, 2 feet deep and 8 feet long was filled with about 600 liters of well water containing trace amounts of naturally occurring minerals. Three kilograms of silver granules prepared by melting a 99.9 purity silver bars in a gas furnace and pouring onto a stainless steel 60 mesh screen placed over a stainless steel drum filled with water. The silver was placed in a 6 cm diameter clear plastic percolation column. Three kilograms of commercially manufactured S. Cerevisiae was added to the tank. The tank reactor was heated to about 40 degrees centigrade with an immersion stainless heater and the aqueous solution was pumped to the top of the percolation column with a submersible pump at the rate of about 40 liters per minute.

The S. Cerevisiae was allowed to mutate for a period of about 30 days until the population density of mutated microbes reached about 3% to 5% by weight. The mutant microbes in the tank reactor were observed under the SEM to have concentric rings of metal within the cellular structure. Under the optical microscope the mutant microbes appeared rod shaped with a flatten bottom on one side. The size was about 1 to 10 micron. After the Sacchromyces had completely mutated and/or died, about 500 grams cane sugar was added as nutrient for the mutant microbes.

After about 30 days, a 10 gram sample of the microbial solution was dried in a desiccator with a vacuum pump for 18 hours. The residue in the desiccator weighed 0.5 grams. A ten gram sample of silver granules coated with the yellow metal was heated in a kiln at about 320 C. After one hour, the yellow metal had vaporized and the silver no longer had a yellow color. The silver granules were then cooled and weighed. After heating, the weight of silver granules decreased in weight by 6 mg. Based on this test, the maximum amount of yellow metal was in the range of 6 parts in 10,000 parts.

A 48 gram sample of silver granules coated with the yellow metal was placed in an Erlenmeyer flask and with 100 ml of 1 N nitric acid solution. The flask was heated on a hot plate until the nitric acid solution reached about 60 C. After about 30 minutes, a small amount of grey-black material was observed in the nitric acid solution and the silver granules no longer had a yellow color. The nitric acid was decanted from the silver granules and filtered onto a filter paper circle placed on sinister glass filtration unit under vacuum. The silver granules in the Erlenmeyer flask after decanting the nitric acid solution was dried and found to weigh 47.9 grams. The nitric acid solution was saved for processing as described below.

The filter paper and the grey-black material were washed with distilled water and placed on about 9 grams of a lead sheet, dried, folded and placed into a bone ash cupel. The cupel was heated at 1800 F for about 30 minutes. On cooling, the cupel had a pale yellow bead weighing about 3 milligrams. The yellow bead was examined on a scanning electron microscope. The spectrum showed silver and gold peaks.

The fire assaying and cupellation methods used above are standard metallurgical methods for precious metals. Fire assaying and cupellation are described by C. W. Ammen, Recovery and Refining of Precious Metals, second edition 1993, Chapter 12, pp 302-329.

Crooks Process. The nitric acid solution/filtrate was placed in a beaker and heated to dryness at 200 C. A white and gray residue was formed in the bottom of the beaker. The beaker was then heated to about 280 C until the white crystals melted to a clear liquid. Distilled water was then added. The grey material which did not dissolve in the water was filtered onto a filter paper circle placed on sinister glass filtration unit under vacuum. The filter paper and grey material were washed with distilled water and placed on about 10 grams of a lead sheet, dried, folded and heated and placed into a bone ash cupel. The cupel was heated at 1800 F for about 30 minutes. On cooling, the cupel had a pale yellow bead weighing about 3 milligrams. The yellow bead was examined on a scanning electron microscope. The spectrum showed silver and gold peaks.

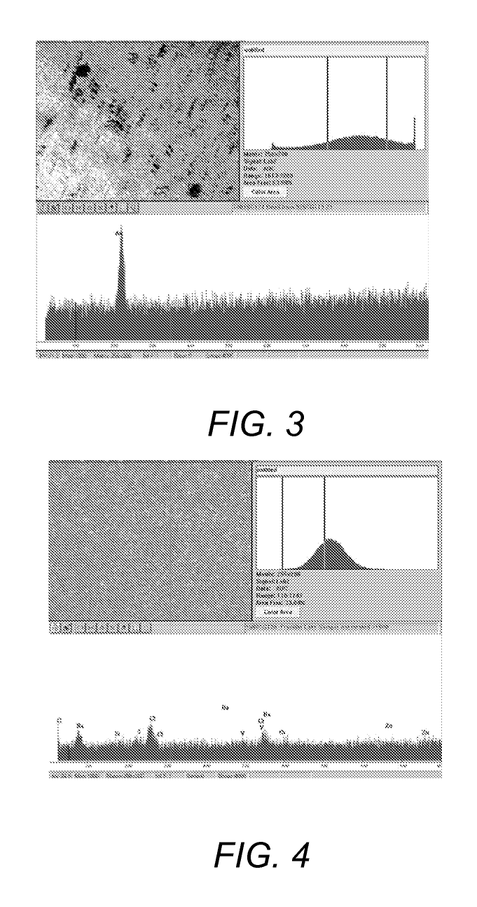

A silver granule coated with yellow material produced by the method of Example 2 was examined with a scanning electron microscope. The spectrum showed a major peak for gold, as is shown in FIG. 3.

### Example 3

### Silver and UV Mutation

A 37 liter glass tank was filled with 12 liters distilled water, 100 grams of 100 micron to 1 millimeter silver particles, 500 grams of dry active Saccharomyces cerevisiae. The tank was maintained at about 25 degrees centigrade and agitated with a small fish aquarium pump and an air stone. The tank was exposed to an ultraviolet mercury lamp of 50 watts. After about five to nine days the microbe density was about 3 to 4% by weight.

The mutant microbes were analyzed with an Induced Coupling Plasma-Mass Spectrometer. Small amounts of silver and gold are detected.

### Example 4

### Silver Mutation

A 37 liter glass tank was filled with 12 liters distilled water, 100 grams of 100 micron to 1 millimeter silver particles, 1400 grams of the wet form of Saccharomyces cerevisiae. The tank was maintained at about 39 degrees centigrade and agitated with a fish aquarium air pump and an air stone. After about 5 days the microbe density was about 3 to 4% by weight and the silver granules were coated with a thin layer of a yellow material.

### Example 5

### Silver Mutation In Salt Water

A 37 liter glass tank was filled with 12 liters distilled water, 100 grams of 100 micron to 1 millimeter silver particles, 1400 grams of the wet form of Saccharomyces cerevisiae and 12 grams of sea salt. The tank was maintained at about 39 degrees centigrade and agitated with a fish aquarium air pump and an air stone. After about 7 days the microbe density was about 3 to 4% by weight and the silver granules were coated with a thin layer of a yellow material. The microbes were moderately active.

A one liter solution of the microbial solution prepared in Example 5 was heated to 95 C on a hot plate. After two days, the microbial density of the microbial solution was about 3 to 4% by weight and the mutant microbes were moderately active.

### Example 6

### Mutation In Electromagnetic Field

A 2-liter beaker was tightly wrapped with 125 feet of 14 gauge insulated copper wire. The ends of the wire were connected to an extension cord and plugged into a Superior Electric variable transformer. The beaker was filled with 100 grams of 99.9% casting silver granules of 1 mm to 10 mm, 100 grams of Saccharomyces cerevisiae and 1000 ml of distilled water. Transformer was adjusted for 7 to 7.5 amps of current through the copper wire to create a magnetic field in the beaker. The temperature of the microbial solution varied from about 35 C to 39 C. An air pump and air stone was used to agitate and to provide air to the microbial solution. Distilled water was added as needed to maintain the microbial solution at about 1000 ml. After about five days, the microbe density was in the range of 1% to 3% by weight and the silver granules were coated with a thin layer of a yellow material.

### Example 7

### Mutation In Electromagnetic Field and Carbon Arc

A beaker wrapped with copper wire as described in Example 6 was equipped with two ⅜ inch diameter by 12 inch long carbon rods. The carbon rods were wired to an extension cord and plugged into a Superior Electric variable transformer. The beaker was filled with 100 grams of 99.9% casting silver granules of 1 mm to 10 mm, 100 grams of Saccharomyces cerevisiae and 1000 ml of distilled water. The transformer was adjusted for 7 to 7.5 amps current through the copper wire to create a magnetic field in the beaker. The second transformer was adjusted to provide about 10 volts to the carbon arcs. The temperature of the microbial solution varied from about 40 C to 45 C. An air pump and air stone was used to agitate and to provide air to the microbial solution. After about 5 days, the microbe density was in the range of 1% to 3% by weight and the silver granules were coated with a thin layer of a yellow material.

### Example 8

### Mutation In Electromagnetic Field and Iron Arc

A beaker wrapped with copper wire as described in Example 6 was equipped with two ⅜ inch by 18 inch iron rods. The iron rods were wired to an extension cord and plugged into a Superior Electric variable transformer. The beaker was filled with 100 grams of 99.9% casting silver granules of 1 mm to 10 mm, 100 grams of Saccharomyces cerevisiae and 1000 ml of distilled water. Transformer was adjusted for 7 to 7.5 amps current through the copper wire to create a magnetic field in the beaker. The second transformer was adjusted to provide about 8 to 10 volts to the iron arcs. The temperature of the microbial solution varied from about 40 C to 45 C. An air pump and air stone was used to agitate and to provide air to the microbial solution. Distilled water was added as needed to maintain the microbial solution at about 1000 ml. After about five days, the microbe density was in the range of 1% to 3% by weight and the silver granules were coated with a thin layer of a yellow material.

### Example 9

### Colloidal Silver Mutation

A 500 ml beaker was filled with 200 ml of distilled water, 10 grams of Saccharomyces cerevisiae and 50 ml of a 10 ppm colloidal silver solution. The beaker was maintained at about 35 C and agitated about every 24 hours with a glass stirring rod. After about 7 days, observation with an optical microscope showed a few live mutant microbes and no live S. cerevisiae. About two grams of silver granules were added to the solution. After about 3 days at a temperature of about 40 C, the silver granules were coated with a pale yellow material.

### Example 10

### Silver Bars

A ten ounce Engelhard 99.9 silver bar was placed in the bioreactor of Example 4. After about ten days, the silver bar was coated a light yellow color with nano gold particles.

### Mineral Ores and Organic Sedimentary Matter Examples

### Example 11

### Digestion Test Lakebed Ore

A lakebed ore from the Franklin Lake alkali playa, Inyo County, Calif. was used in this test. 50 g of the ore milled to about 100 mesh, 100 ml of the microbe prepared in Example 4 and 100 ml of distilled water were placed into a 50 flat bottom Florence flask. The flask was stirred with a magnetic stir bar and heated to 50 C for three days. The microbial solution was assayed by the HP 4500 ICP-MS. A two gram sample of the ore residue/solids was placed in aqua regia (one part nitric acid and three parts hydrochloric acid) at about 20 C.

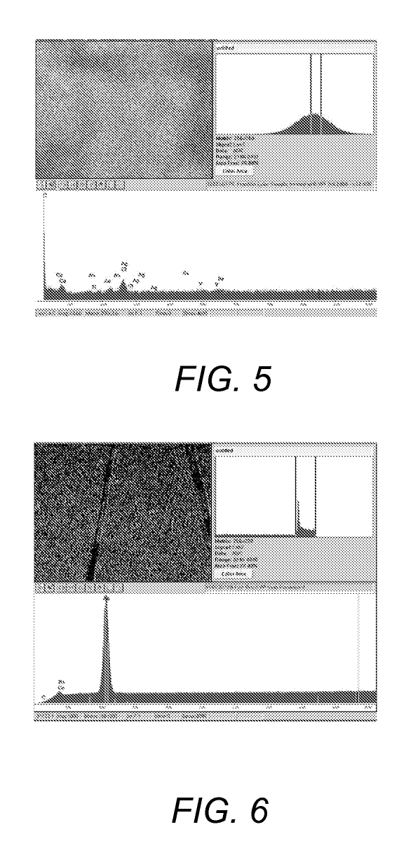

A sample of the ore used in Example 11 was examined with a scanning electron microscope and the results are shown in FIG. 4. As is shown, the spectrum showed no silver and gold peaks. Nevertheless, the aqua regia solution was analyzed with the HP ICP-MS, and gold, silver and palladium in the amount of 10 ppm to 100 ppm were detected in the aqua regia solution. A sample of the ore after biotreatment with mutant microbes for 3 days by the method of Example 11 was dried and examined with a scanning electron microscope. The resulting spectrum, shown in FIG. 5, showed silver and gold peaks.

### Example 12

### Digestion Test Arizona Ore

The mutant microbes prepared by the method of Example 4 were used to digest a gypsiferous mineral ore of red mudstone and siltstone with thin-bedded to laminated gypsum and green mudstone from during the Tertiary period from the Tonto Basin area of Arizona. The digestion procedure was carried out according the procedure of Example 11 or three days. The microbial solution was assayed by the HP 4500 ICP-MS. A two gram sample of the ore residue/solids was placed in aqua regia (one part nitric acid and three parts hydrochloric acid) at about 20 C. The aqua regia solution was analyzed with the HP ICP-MS, and gold, silver and palladium in the amount of 10 ppm to 100 ppm were detected in the aqua regia solution.

### Example 13

### Digestion Test-Oil Shale

This test used oil shale from the Green River Formation of Wyoming and Colorado. A 50 g sample of the shale milled to about 100 mesh, 100 ml of the microbe prepared by the method of Example 4 and 100 ml of distilled water were placed into a 50 flat bottom Florence flask. The flask was stirred with a magnetic stir bar and heated to 80 C for three days. The microbial solution was assayed by the HP 4500 ICP-MS and the solution was found to contain about 10 ppm silver.

### Example 14

### Digestion Test-Flotation Concentrates of Arsenosulfide Ore

A flotation concentrate having about 30 ppm gold was used in this test. The concentrate was prepared from an arsenosulfide ore from the Shandong Province of China. A 50 g sample of the concentrate, 100 ml of the microbe prepared by the method of Example 4 and 100 ml of distilled water were placed into a 50 flat bottom Florence flask. The flask was stirred with a magnetic stir bar and heated to 50 C for three days. A two gram sample of the ore residue/solids was placed in aqua regia (one part nitric acid and three parts hydrochloric acid) at about 20 C. The aqua regia solution was analyzed with the HP ICP-MS, and trace amounts of silver were detected in the aqua regia solution.

### Example 15

### Digestion In Electromagnetic Field

A beaker wrapped with copper wire as described in Example 6 was filled with 1000 ml of mutant microbes prepared by the method of Example 4, 50 ml of nitric acid (68%) and 100 grams of the ore used in Example 14. Transformer was adjusted for 7 to 7.5 amps through the copper wire to create a magnetic field in the beaker. The temperature of the microbial solution varied from about 40 C to 45 C. An air pump and air stone was used to agitate and to provide air to the microbial solution. After a few days, a one milliliter aliquot of the microbial solution was dried on a 8 gram sheet of assay lead formed in the shape of a boat. The lead was folded and placed in a bone ash cupel and placed into an electric kiln at about 1800 F. After about 30 minutes a small silvery metal bead was produced in the cupel.

### Example 16

### Digestion Test-Flotation Tails

The tails from a flotation concentrate having about 1 ppm gold was used in this test. A 50 g sample of the tails, 100 ml of the microbe prepared by the method of Example 4 and 100 ml of distilled water were placed into a 50 flat bottom Florence flask. The flask was stirred with a magnetic stir bar and heated to 50 C for three days. A one gram sample of the ore residue/solids was placed in aqua regia (one part nitric acid and three parts hydrochloric acid) at about 20 C. Trace amounts of silver was detected with the ICP-MS.

### Example 17

### Digestion Test on Vernal Oil Shale

A 500 g (100 mesh) sample of oil shale from Vernal, Utah (Bureau Land Management stockpile for research testing), 1000 ml of mutant microbe solution prepared by the method of Example 4 with about a 3% microbe density by weight was contacted in a 1500 ml open beaker at a temperature of about 80 C. The digestion mixture was stirred periodically with a glass stirring rod. After six hours, the mixture was allowed to settle. The shale settled to the bottom of beaker. On top of the shale was a thin layer of oil products released from the shale. On top of the oil layer was the aqueous microbial solution. The beaker was stirred periodically for another 48 hours at a temperature of about 80 C. After the additional digestion time, the mixture was allowed to settle. The shale residue settled to the bottom. The next layer was the microbial aqueous solution. The organic layer was on top of the aqueous solution.

### Example 18

### Digestion Test on Tar Sands

A 50 g sample of tar sands from the Athabasca deposit in Alberta, Canada and 200 ml of the microbial solution prepared by the method of Example 4 were placed in 500 ml beaker. The beaker was agitated with a fish aquarium pump and air stone and heated to 60 C. on a hot plate. After about 5 days at 60 C, the tar was released from sands leaving a mixture of light grey sand and tar in the microbial solution. When the beaker was heated at 80 C for 24 hours, the tar became a light oil that floated to the top of the microbial solution.

### Precious Metal Production Examples

### Example 19

### Metal Recovery with Nitric Acid

A 2-liter beaker was filled with 1500 ml of microbial solution prepared by the method of Example 4, 100 grams of silver granules and 50 ml of nitric acid (68%). The beaker was heated on a hot plate and stirred with a magnetic stir bar. After about 3 days at a temperature of about 39 C, 96 grams of silver was removed from the beaker. The amount of silver dissolved by the nitric acid was about 4 grams. The microbial solution was then allowed to evaporate to dryness in the beaker at about 100 C. The brown/yellow organic residue was wrapped in about 100 g of lead sheet and placed in a cupel. The cupel was heated at 1800 F in an electric kiln. After one hour, a 7 gram silver bead was obtained.

### Example 20

### Metal Recovery with Nitric Acid

A 2-liter beaker was filled with 1500 ml of microbial solution prepared by the method of Example 4 and 50 ml of nitric acid (68%). The beaker was heated on a hot plate and stirred with a magnetic stir bar. After about 3 days at a temperature of about 35 C, the beaker was stirred and agitated with a glass stirring rod and a 5-ml aliquot of the microbial mixture was removed. The 5 ml aliquot was placed in 20 grams of lead sheet folded to the shape of a boat. The boat was heated at about 150 C to dryness, folded and placed in a bone ash cupel. The cupel was heat was heated at 1800 F in an electric kiln. After one hour, a small silvery metal bead was obtained.

### Example 21

### Metal Recovery in Electromagnetic Field and Carbon Arc

A beaker wrapped with copper wire described in Example 6 was equipped with two ⅜ inch by 18 inch carbon rods. The carbon rods were wired to an extension cord and plugged into a Superior Electric variable transformer. The beaker was filled with 50 grams of 99.9% casting silver granules of 1 mm to 10 mm, 1000 milliliters of microbial solution prepared by the method of Example 4. Five milliliters of nitric acid (70%) was added. Transformer was adjusted for 7 to 7.5 amps current through the copper wire to create a magnetic field in the beaker. The second transformer was adjusted to provide about 8 to 10 volts to the carbon arcs. The temperature of the microbial solution varied from about 40 C to 45 C.

After about 30 days, the microbial mixture of live and dead microbes was decanted from the solid silver granules. After washing with water and drying, the recovered silver granules weighed 100 grams. The microbial mixture then was allowed to slowly evaporate at about 25 C. After a period of about 45 days, the microbial mixture produced a black biomass of dead microbes weighing about 100 grams. A two gram sample of the biomass was placed into about 10 grams of lead sheet folded in the shape of boat. The lead was folded and placed in a bone ash cupel and heated in a kiln at 1800 F for one hour. A silver bead weighing about 0.2 gram was produced.

### Example 22

### Metal Recovery in Electromagnetic Field

A beaker wrapped with copper wire as described in Example 6 was filled with 1000 milliliters of microbial solution prepared by the method of Example 4. Transformer was adjusted for 7 to 7.5 amps through the copper wire to create a magnetic field in the beaker. The temperature of the microbial solution varied from about 35 C to 39 C. An air pump and air stone was used to agitate and to provide air to the microbial solution. After a period of about 30 days, the microbial solution was allowed to evaporate to produce a black biomass of dead microbes weighing about 50 grams. A five gram sample of biomass was placed into about 20 grams of a lead sheet folded in the shape of boat. The lead folded and placed in a bone ash cupel and heated in a kiln at 1800 F. A 10 mg silver bead was produced.

### Example 23

### Metal Recovery from Microbial Solution

A microbial solution prepared by the method of Example 4 was maintained at about 25 C for 30 days. A 2 ml sample of the microbial solution was placed into a clay scarifying dish and evaporated. The dish was then placed into an electric kiln with tungsten elements and heated at about 320 C for 14 hours. About 10 grams of lead sheet was added and the dish heated to about 980 C. The molten lead and slag was then poured into a cone mold. The lead was separated and pounded into a cube. The lead cube was placed into a bone ash cupel and heated at 980 C to give 5 mgs of a silvery bead with a light yellow color.

### Example 24

A quart jar with a metal lid was filled with 500 ml of distilled water, 7 grams of Sacchromyces Cerevisiae and 10 grams of silver granules of about 1 mm to 5 mm. The jar was loosely covered with the lid and heated on a hot plate to bring the solution temperature to about 35 C. After about 5 days, the silver was coated a pale yellow color with a yellow material. The observation of the microbial solution with an optical microscope showed that the mutant microbe density was about 1%.

### Example 25

A second test in a quart jar was done as described in Example 24. All reaction conditions and materials were identical except that an air stone was used to pump air into the bottom of the jar. After about 5 days, the silver was coated a yellow color that was visually observed to be more yellow than the Example 24. Also, the observation of microbial solution with an optical microscope showed that the mutant microbe density was about 2%.

### Example 26

A 500 ml sample of the microbial solution prepared by the method of Example 4 having a mutant microbe density of about 3% was placed in a beaker with 20 grams of silver granules sized about 1 mm to 5 mm. The solution was heated at 39 C. After about 4 hours, the silver was observed to have a yellow coating.

### Example 27

A 500 ml sample of the same microbial solution used in Example 26 was placed in a beaker with 20 grams of silver granules sized about 1 mm to 5 mm. The solution was heated at 80 C. After about two hours, the silver granules were coated with a yellow color.

### Example 28

A 500 ml sample of the same microbial solution used in Example 26 was placed in a beaker with ten (10) grams of sea salt. The microbial solution was heated to 90 C. for 24 hours. Observation of the microbial solution after cooling with an optical microscope showed the microbial solution had a mutant microbe density of about 3 percent that was moderately active.

### Example 29

After 90 days, the contents of the microbial tank of Example 2 was evaporated to dryness at about 25 C to 30 C to give a biomass of dead microbes. The biomass was examined with a scanning electron microscope. FIG. 6 is an SEM image showing the spectrum which indicates a major peak for gold.

Methods for producing mutant microbes that coat silver with a yellow metal and uses of the mutant microbes for recovering precious metals and producing biofuels and oil products have been described in the accordance with the embodiments shown. The mutant microbes are particularly useful for industrial applications because they survive high temperatures and highly acidic and basic environments.

One of ordinary skill in the art will readily recognize that there could be variations to the embodiments, and any variation would be within the spirit and scope of the present invention. Accordingly, many modifications may be made by one of ordinary skill in the art without departing from the spirit and scope of the appended claims.

What is claimed is:

1. A mutant microbe used for generating trace amounts of gold particles on metallic silver, the mutant microbe produced by placing metallic silver in an aqueous solution, and adding a species of Saccharomyces to the aqueous solution such that when the species of Saccharomyces comes in contact with the metallic silver, at least a portion of the species of Saccharomyces transforms into the mutant microbe that interacts with the metallic silver and forms a layer comprising a trace amount of nano gold particles on the metallic silver.

2. The mutant microbe of claim 1 wherein the silver is from 1 micron particles to silver bars.

3. The mutant microbe of claim 1 wherein the species is Saccharomyces cerevisiae.

4. A method of producing a mutant microbe used for generating trace amounts of gold particles on metallic silver, the method comprising:
placing metallic silver in an aqueous solution; and
adding a species of Saccharomyces to the aqueous solution such that when the species of Saccharomyces comes in contact with the metallic silver, at least a portion of the species of Saccharomyces transforms into a mutant microbe that interacts with the metallic silver and forms a layer comprising a trace amount of nano gold particles on the metallic silver.

5. The method of claim 4 wherein the silver is from 1 micron particles to silver bars.

6. The method of claim 4 wherein the species is Saccharomyces cerevisiae.

7. A method of producing precious metals, the method comprising:
placing metallic silver in an aqueous solution;
adding a species of Saccharomyces to the aqueous solution such the when the species of Saccharomyces comes in contact with the metallic silver, at least a portion of the species of Saccharomyces transforms into a mutant microbe that includes clusters of precious metal atoms within its cytoplasm and forms a layer comprising a trace amount of nano gold particles on the metallic silver; and
recovering the cluster of precious metal atoms from the mutant microbe.

8. The method of claim 7 wherein recovering the cluster of precious metal atoms includes extracting the precious metal atoms from biomass of dead mutant microbes.

9. The method of claim 8 wherein the precious metal is silver.

10. A method of producing precious metals, the method comprising:
placing metallic silver in an aqueous solution;
adding a species of Saccharomyces to the aqueous solution such the when the species of Saccharomyces comes in contact with the metallic silver, at least a portion of the species of Saccharomyces transforms into a mutant microbe that interacts with the metallic silver and forms a layer comprising a trace amount of nano gold particles on the metallic silver; and
contacting a mineral ore with an aqueous solution including the mutant microbe.

11. The method of claim 10 wherein the silver is from 1 micron particles to silver bars.

12. The method of claim 10 wherein the species is Saccharomyces cerevisiae.

13. A method of producing oil products from at least one of a sedimentary organic rock, heavy oil and a biomass, the method comprising:
placing metallic silver in an aqueous solution;
adding a species of Saccharomyces to the aqueous solution such the when the species of Saccharomyces comes in contact with the metallic silver, at least a portion of the species of Saccharomyces transforms into a mutant microbe that interacts with the metallic silver and forms a layer comprising a trace amount of nano gold particles on the metallic silver; and
contacting at least one of the sedimentary organic rock, the heavy oil and the biomass with the mutant microbe.

14. The method of claim 13 wherein the sedimentary organic rock is at least one of oil shale and oil sands.

15. The method of claim 13 wherein the species is Saccharomyces cerevisiae.

16. The method of claim 13 in which the biomass is dead mutant microbes.
